(19) United States
(12) Patent Application Publication
(10) Pub. No.: US 2021/0045832 A1
(43) Pub. Date: Feb. 18, 2021

(54) Low Cost Sharps Container Using Recycled Components

(71) Applicant: Kelce S. Wilson, Murphy, TX (US)

(72) Inventor: Kelce S. Wilson, Murphy, TX (US)

(21) Appl. No.: 17/086,433

(22) Filed: Nov. 1, 2020

Related U.S. Application Data
(60) Provisional application No. 62/931,497, filed on Nov. 6, 2019.

(51) Int. Cl. A61B 50/36; B09B 3/00; A61L 31/04

(52) U.S. Cl. CPC A61B 50/362; A61L 31/04; B09B 3/0075

(57) ABSTRACT

A special bottle lid renders ubiquitous pill bottles into safe, effective, low cost sharps containers. For example, a common plastic bottle, such as one that holds 120 capsules, each sized for 1 gram of material, is conveniently sized to make a sharps container suitable for holding needles (detached from syringes) and blood test lancets used by diabetics. As a current bottle becomes filled, the special lid is moved to a second bottle, and the lid from the second bottle is used to seal the first bottle for disposal. This repeats as each new bottle is filled. Thus, the primary investment is the special lid, which may be reused with new empty bottles indefinitely. Example bottle dimensions range from 2.5 to 3 inches in diameter and 4.5 to 5.5 inches in height. Syringes without the needles (and thus safer) may be placed in a different, larger container.

### CROSS REFERENCE TO RELATED APPLICATIONS

This application claims the benefit of U.S. Provisional Patent Application No. 62/931,497, filed Nov. 6, 2019, entitled “Low Cost Sharps Container Using Recycled Components”, the entirety of which is hereby incorporated by reference herein.

### BACKGROUND

Sharps containers are typically used for disposal of used needles and blood test lancets, including for self-care solutions performed in consumers' homes. The use of sharps containers may reduce the potential dangers of placing sharp objects in normal household trash. People having certain medical conditions that warrant on-going blood tests and injections, such as diabetics, thus have a need for a supply of sharps containers at their homes. Unfortunately, a sharps container then becomes yet another medical supply item that must be acquired by people who are typically burdened with acquiring a myriad of other medical supplies.

### BRIEF DESCRIPTION OF THE DRAWINGS

The disclosed examples are described in detail below with reference to the accompanying drawing figures listed below:

FIG. 1 illustrates an exemplary inventive sharps container;

FIG. 2 illustrates another view of the sharps container of FIG. 1;

FIG. 3 illustrates a lower portion of a lid for the sharps container of FIG. 1;

FIG. 4 illustrates an upper portion of a lid for the sharps container of FIG. 1;

FIG. 5 illustrates a second lid and a second bottle that, together with the sharps container of FIG. 1, may form an exemplary sharps disposal system;

FIG. 6 illustrates an exemplary sharps container warning label for the sharps container of FIG. 1; and

FIG. 7 shows a flow chart of exemplary operations associated with the sharps container of FIG. 1.

### DETAILED DESCRIPTION

Serendipitously, the people in greatest need of sharps containers may often have the materials readily on-hand that are needed for assembling low cost sharps containers: empty pill bottles. Empty pill bottles, along with a single special lid that is transferred to each new bottle as it is used to receive sharp objects, become sharps containers that use recycled components. The regular pill bottle lids are then used to seal the full bottles for final disposal. Not only does this save users time and money, by avoiding the need to purchase traditional sharps containers, but it is ecologically friendly because it recycles pill bottles and lids into components of an inventive sharps disposal system.

As is disclosed herein, a single special bottle lid, along with a set of labels, can function to render ubiquitous pill bottles into safe, effective, low cost sharps containers. For example, a conveniently-sized plastic bottle that is common, such as may be used for holding 120 capsules of fish oil (or some other substance), each sized at 1 gram (1,000 milligrams), is useful in many scenarios. Example bottle dimensions range from 2.5 to 3 inches in diameter and 4.5 to 5.5 inches in height. Other sizes can also be acceptable. A set of labels that can be affixed to the bottles can be furnished inexpensively, or printed on demand with home printers. A single special lid provides an easily-operated, safe access port for rapidly inserting used needles, lancets, or other sharp objects, and which may be closed to prevent spillage.

When a bottle is to be used as the current sharps container, its original lid is set aside or used to seal an earlier-used (and now full) bottle, and the special lid is attached to the bottle. Needles, lancets, and even blood test strips, can be securely deposited, safe from accidental spillage, while the special lid remains on the bottle. Upon that bottle's sharp object content nearing capacity, the special lid is removed. The original lid is replaced, or a lid from a newer bottle is used, and the special lid is moved to the new bottle. Thus, only an investment in a single special lid is needed (and perhaps labels), because the special lid is transferred to each bottle as that bottle is becomes the current sharps receptacle. The original, unmodified lids are used to seal the full bottles for final disposal.

Labels can be affixed to the bottles, as they are moved through process of being advantageously recycled into the sharps containers, to provide any necessary warnings against opening full bottles. Glue and tape may also be used to create a physical deterrent to removing a lid of a bottle filled with sharp objects. Labels can be pre-printed adhesive labels, peeled from a backing layer and affixed to the bottles, or printed by the user and taped or glued to the bottles. To preserve the volume of the pill bottles for the most potentially dangerous items (e.g., needles, lancets, and test strips having deposited blood), users can remove needles from syringes and dispose of the syringes (without the needles) in another, larger container, such as an empty laundry fabric softener bottle or another similarly-sized and sturdy container.

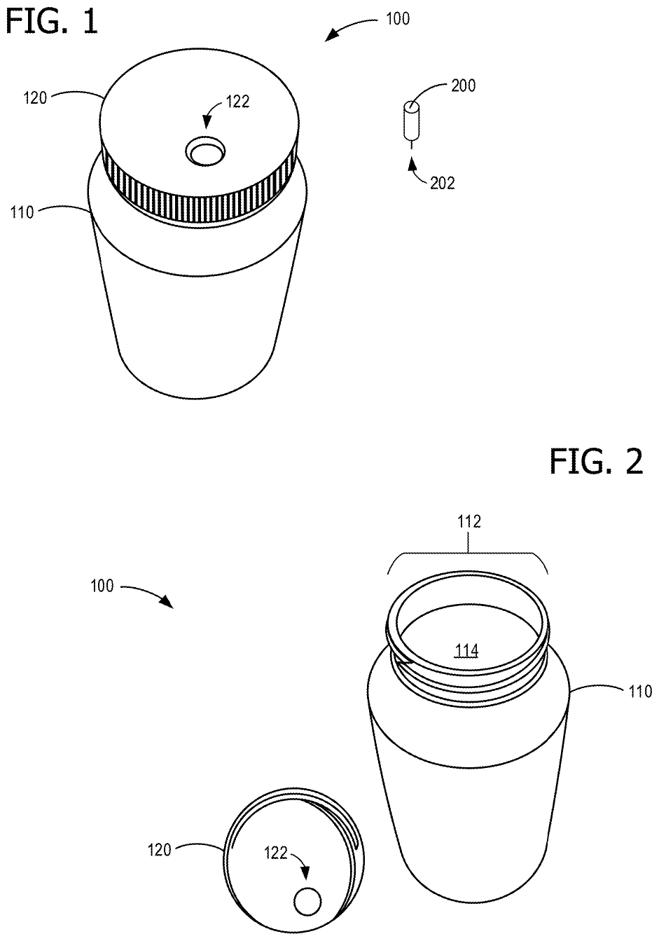

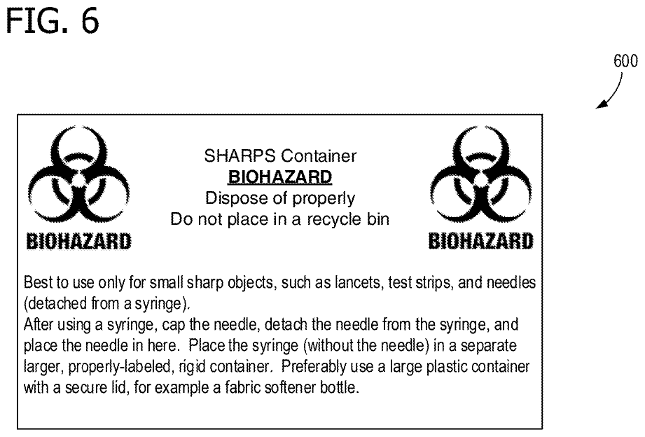

FIG. 1 illustrates an exemplary sharps container 100 comprising a bottle 110 and a lid 120. An exemplary sharps disposal system includes at least sharps container 100, and may also include one or more additional lids and one or more additional bottles. See FIG. 5 and its description for more details regarding additional lids and bottles. See FIG. 6 for an example sharps container warning label that may be affixed to bottle 110. In some examples, bottle 110 is a common plastic pill bottle, such as may be used for holding 120 capsules of fish oil (or some other substance), each sized at 1 gram (1,000 milligrams). Such bottles, and similarly-sized bottles, are common, and thus provide a low-cost, easily-replenished supply of replaceable container components. An example sharp object 200 is illustrated as a blood test lancet, such as may be used by diabetics for blood glucose testing. Sharp object 200 has a needle 202 that presents a hazard, if sharp object 200 were to be placed in normal trash receptacles without the protection of a suitable sharps container.

Bottle 110 is shown in additional detail in FIG. 2. Bottle 110 has an interior 114 and a threaded aperture 112. In some examples, bottle 110 is comprised of plastic. In some examples, bottle 110 is opaque. In some examples, threaded aperture 112 of bottle 110 has a diameter of 1.75 to 2.5 inches. In some examples, interior 114 of bottle 110 has a volume of 20 to 40 cubic inches.

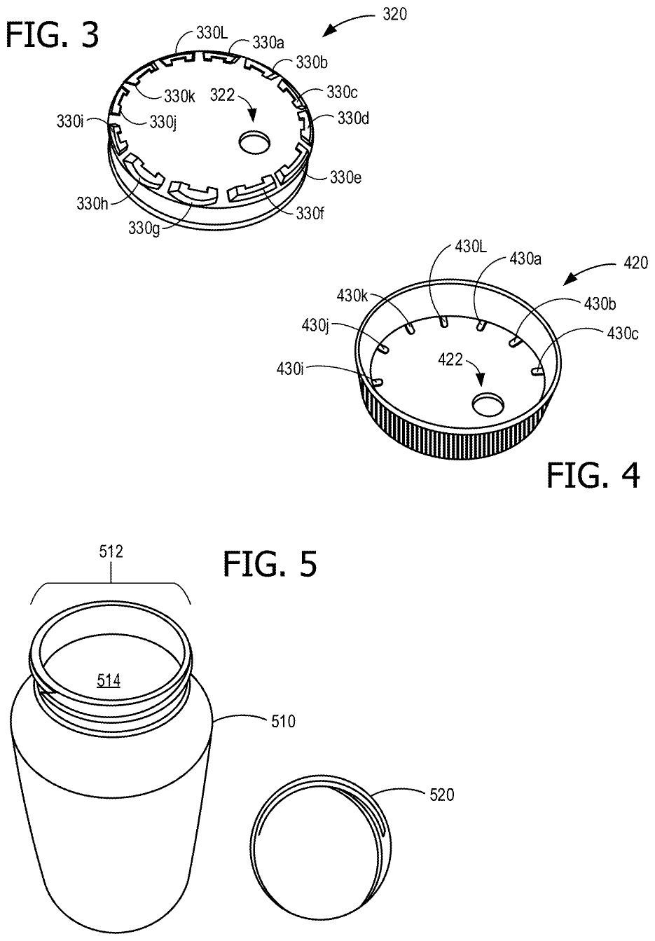

Lid 120 is shown in additional detail in FIGS. 2, 3 and 4. As illustrated in FIG. 2, lid 120 has an off-center aperture 122. Lid 120 has a lower portion 320, shown in FIG. 3, and an upper portion 420, shown in FIG. 4. In the illustrated example, lid 120 is configured as a ratcheting safety lid. Lower portion 320 is sized and threaded to mate with threaded aperture 112 of bottle 110, and has an off-center aperture 322. Upper portion 420 similarly has an off-center aperture 422. Upper portion 420 and the lower portion 320 are arranged within lid 120 to enable upper portion 420 and lower portion 320 to rotate in one direction relatively to each other, such that aperture 422 of upper portion 420 can rotate to align with aperture 322 of lower portion 320. When aligned, aperture 422 and aperture 322 together form aperture 122 passing completely through lid 120. When lid 120 is on (e.g., fully screwed onto) bottle 110, aperture 122 provides access to interior 114 of bottle 110. This enables a user to deposit sharp object 200 into interior 114 of bottle 110.

However, aperture 422 of upper portion 420 can rotate away from alignment with aperture 322 of lower portion 320 to close off access to interior 114 of bottle 110. This rotation closes aperture 122, preventing accidental spillage of sharp object 200 and other sharp objects within bottle 110. In some examples, aperture 322 of lower portion 320 has a maximum dimension of 0.25 to 0.75 inches. In some examples, aperture 322 is circular, and its maximum dimension is its diameter. In some examples, aperture 322 is oval, rectangular, or another shape. In some examples, aperture 422 of upper portion 420 has a maximum dimension of 0.25 to 0.75 inches. In some examples, aperture 422 is circular, and its maximum dimension is its diameter. In some examples, aperture 422 is oval, rectangular, or another shape. In some examples, apertures 322 and 422 have the same maximum dimension.

In some examples, lid 120 is based on a common ratcheting safety lid, as is common medicine bottles as a child safety feature. In some examples, lid 120 comprises a set of ramped ratchet teeth 330a-330L (on lower portion 320) and a set of cogs 430a-430L (on upper portion 420). Cogs 430d-430h are hidden from view, and thus not labeled in FIG. 4. Upper portion 420 and lower portion 320 are arranged so that set of ramped ratchet teeth 330a-330L and set of cogs 430a-430L engage to permit lid 120 to be screwed onto bottle 110. That is, set of ramped ratchet teeth 330a-330L engages set of cogs 430a-430L to prevent upper portion 420 from rotating relative to lower portion 320 when rotational force is applied in a direction that screws lid 120 onto bottle 110.

However, upper portion 420 and lower portion 320 are arranged so that set of ramped ratchet teeth 330a-330L and set of cogs 430a-430L permit upper portion 420 to rotate relatively to lower portion 320 without lid 120 being unscrewed from the first bottle. This free rotation prevents lid 120 from being removed from bottle 110, unless a downward compression force is applied to upper portion 420. In this manner, compression of upper portion 420 against lower portion 320 permits lid 120 to be unscrewed from bottle 110. In some examples, lid 120 is configured so that a sideways compression force, that slightly deforms upper portion 420 from a circular shape into an oval shape, is what permits lid 120 to be unscrewed from bottle 110.

Exemplary operation of lid 120 takes three forms: (1) A clockwise rotational force applied to lid 120 screws lid 120 onto bottle 110. (2) A counter-clockwise rotational force, without a downward compression force on upper portion 420, rotates upper portion 420 relative to lower portion 320 so that apertures 322 and 422 either align and open aperture 122 (to permit sharp object 200 to be deposited into interior 114 of bottle 110) or close off aperture 122 (thereby sealing sharp object 200 within interior 114 of bottle 110). (3) A counter-clockwise rotational force, with a downward compression force on upper portion 420, permits lid 120 to be unscrewed from bottle 110.

FIG. 5 illustrates a second lid 520 and a second bottle 510. Second lid 520 is sized and threaded to mate with threaded aperture 112 of bottle 110. In some examples, second lid 520 is configured as a ratcheting safety lid. In some examples, second lid 120 does not have an aperture that can provide access to interior 114 of first bottle 110, when second lid 120 is on bottle 110. That is, in some examples, second lid 520 is the same size, shape, and configuration as lid 120, but just missing aperture 122. In such examples, lid 120 is a duplicate of second lid 520, but with the addition of aperture 122 (which is a combination of apertures 322 and 422). In such examples, lid 120 can be easily made from a surplus second lid 520 by drilling a single off-center hole through both of the ratcheting portions, while ensuring that the different portions do not rotate relatively to each other during the drilling operation. Or, a lid could be provided with some product that did not have aperture 122 fully open upon a consumer opening a package, such as at least one of apertures 322 and 422 was blocked with easily removable material. For example, the lid could be part of product packaging for retail distribution. However, if there was scoring within the lid, or some other feature that made it easy for consumers to pop out whatever material was the blocking one of apertures 322 and 422 then, upon the consumer using up the contents of the package, the user could easily modify the lid to turn it into lid 120 with aperture 122, and use it as disclosed herein.

In some examples, second bottle 510 has an interior 514 and an aperture 512 sized and threaded to mate with the first lid. In some examples, second bottle 510 is comprised of plastic. In some examples, second bottle 510 is opaque. In some examples, threaded aperture 512 of second bottle 510 has a diameter of 1.75 to 2.5 inches. In some examples, interior 514 of second bottle has a volume of 20 to 40 cubic inches. In some examples, second bottle 510 is the same size as bottle 110, although in some examples, second bottle 510 is taller or shorter than bottle 110. A sharps disposal system can comprise just bottle 110 and lid 120, or may additionally include ore or more of second bottle 510 and/or one or more of second lid 520.

FIG. 6 illustrates an exemplary sharps container warning label 600. Sharps container warning label 600 may be to bottle 110, and second bottle 510, as second bottle 510 replaces bottle 110 as the current sharps receptacle. In some examples, sharps container warning label 600 is affixed to bottles using an adhesive backing. In some examples, sharps container warning label 600 is affixed to bottles using tape or glue. The illustrated sharps container warning label 600 contains the text: “SHARPS Container; BIOHAZARD; Dispose of properly; Do not place in a recycle bin; Best to use only for small sharp objects, such as lancets, test strips, and needles (detached from a syringe). After using a syringe, cap the needle, detach the needle from the syringe, and place the needle in here. Place the syringe (without the needle) in a separate larger, properly-labeled, rigid container. Preferably use a large plastic container with a secure lid, for example a fabric softener bottle.” It should be understood that different text may be used, as well as colors for the text and/or images.

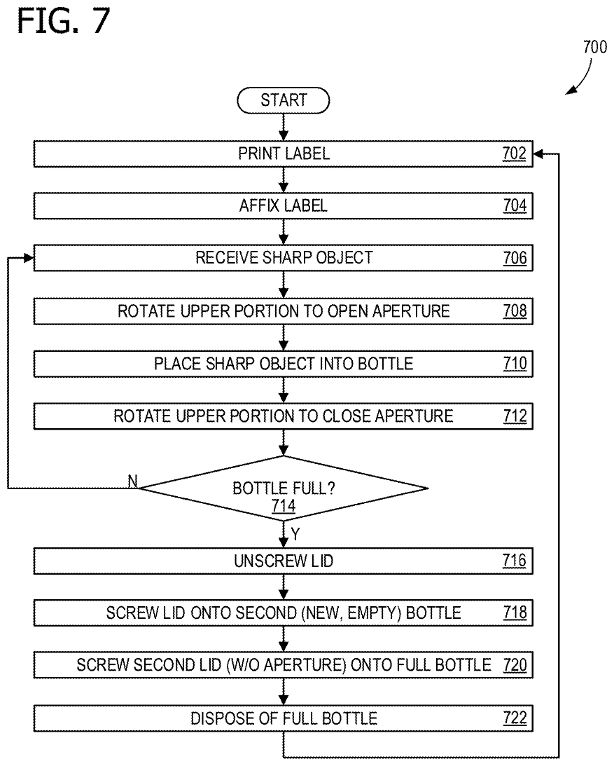

FIG. 7 shows a flow chart 700 of exemplary operations associated with sharps container 100 of FIG. 1. Specifically, flow chart 700 illustrates a method of sharps disposal using a sharps disposal system as disclose herein. Operation 702 includes printing a sharps container warning label, and operation 704 includes affixing the sharps container warning label to a bottle. A sharp object is received at 706. Operation 708 includes rotating an upper portion of a lid relatively to a lower portion of the first lid to provide access to an interior of the bottle onto which the lid is affixed. This opens an aperture that passes entirely through the lid. Operation 710 includes placing the sharp object into the interior of the bottle 110. Operation 712 includes rotating the upper portion relatively to the lower portion to close off access to the interior of the bottle. This closes the aperture.

Decision operation 714 determines whether the bottle is full. For example, a user could remove the lid and look inside, look through the aperture while the lid is still in place, feel the weight of the bottle, or notice difficulty in placing the most recent object within the bottle (e.g., by the friction of the most recent sharp object being forced tightly between previously inserted sharp objects). If the bottle is not full, flow chart 700 returns to operation 706 to receive the next sharp object. If the bottle is full, flow chart 700 moves on to operations 716-720 to transfer the lid onto a new, empty bottle and seal the full bottle with a lid that does not have an aperture.

Specifically, operation 718 includes unscrewing the lid from the full bottle. Operation 718 includes screwing the lid onto a second bottle that is empty and will be used as the new sharps container in the next pass through operations 706-712. Operation 720 includes screwing a second lid onto the prior-used bottle that is now full. The full bottle, with a solid lid (no aperture that could accidentally open) is disposed of at 722. Flow chart 700 then returns to operation 702, which now includes printing another sharps container warning label for the second (new, empty) bottle. Operation 704 then includes affixing the new container warning label to the second bottle. The second bottle is then used with the lid having the aperture to receive additional sharp objects. Flow chart 700 continues on with new bottles and lids as the currently-used bottles become full and are disposed of with solid lids.

Another exemplary sharps disposal system comprises: a first bottle having an interior and a threaded aperture; and a first lid configured as a ratcheting safety lid, the first lid comprising: a lower portion sized and threaded to mate with the threaded aperture of the first bottle, and having an off-center aperture; and; an upper portion having an off-center aperture; wherein the upper portion and the lower portion are arranged to enable the upper portion and the lower portion to rotate in one direction relatively to each other, such that the aperture of the upper portion can rotate to align with the aperture of the lower portion to provide access to the interior of the first bottle, and the aperture of the upper portion can rotate away from alignment with the aperture of the lower portion to close off access to the interior of the first bottle.

An exemplary method of sharps disposal comprises: rotating an upper portion of a first lid relatively to a lower portion of the first lid to provide access to an interior of a first bottle onto which the first lid is affixed; placing a sharp object into the interior of the first bottle; and rotating the upper portion relatively to the lower portion to close off access to the interior of the first bottle.

Another exemplary sharps disposal system comprises: a first bottle having an interior and a threaded aperture, wherein the first bottle is comprised of plastic, wherein the first bottle is opaque, wherein the threaded aperture of the first bottle has a diameter of 1.75 to 2.5 inches, and wherein the interior of the first bottle has a volume of 20 to 40 cubic inches; and a first lid configured as a ratcheting safety lid, the first lid comprising: a lower portion sized and threaded to mate with the threaded aperture of the first bottle, and having an off-center aperture, wherein the aperture of the lower portion has a maximum dimension of 0.25 to 0.75 inches; an upper portion having an off-center aperture, wherein the aperture of the upper portion has a maximum dimension of 0.25 to 0.75 inches; wherein the upper portion and the lower portion are arranged to enable the upper portion and the lower portion to rotate in one direction relatively to each other, such that the aperture of the upper portion can rotate to align with the aperture of the lower portion to provide access to the interior of the first bottle, and the aperture of the upper portion can rotate away from alignment with the aperture of the lower portion to close off access to the interior of the first bottle; wherein the first lid comprises a set of ramped ratchet teeth and a set of cogs; wherein the upper portion and the lower portion are arranged so that the set of ramped ratchet teeth and the set of cogs engage to permit the first lid to be screwed onto the first bottle; wherein the upper portion and the lower portion are arranged so that the set of ramped ratchet teeth and the set of cogs permit the upper portion to rotate relatively to the lower portion without the first lid being unscrewed from the first bottle; and wherein compression of the upper portion against the lower portion permits the first lid to be unscrewed from the first bottle; a second lid sized and threaded to mate with the threaded aperture of the first bottle, wherein the second lid is configured as a ratcheting safety lid and wherein the second lid does not have an aperture that can provide access to the interior of the first bottle; and a second bottle having an interior and an aperture sized and threaded to mate with the first lid, herein the second bottle is comprised of plastic, wherein the second bottle is opaque, wherein the threaded aperture of the second bottle has a diameter of 1.75 to 2.5 inches, and wherein the interior of the second bottle has a volume of 20 to 40 cubic inches.

Alternatively, or in addition to the other examples described herein, examples include any combination of the following:
the first bottle is comprised of plastic;
the first bottle is opaque;
the threaded aperture of the first bottle has a diameter of 1.75 to 2.5 inches;
the interior of the first bottle has a volume of 20 to 40 cubic inches;
the aperture of the lower portion has a maximum dimension of 0.25 to 0.75 inches;
the aperture of the upper portion has a maximum dimension of 0.25 to 0.75 inches;
the first lid comprises a set of ramped ratchet teeth and a set of cogs;
the upper portion and the lower portion are arranged so that the set of ramped ratchet teeth and the set of cogs engage to permit the first lid to be screwed onto the first bottle;
the upper portion and the lower portion are arranged so that the set of ramped ratchet teeth and the set of cogs permit the upper portion to rotate relatively to the lower portion without the first lid being unscrewed from the first bottle;
compression of the upper portion against the lower portion permits the first lid to be unscrewed from the first bottle;
a second lid sized and threaded to mate with the threaded aperture of the first bottle;
the second lid is configured as a ratcheting safety lid;
the second lid does not have an aperture that can provide access to the interior of the first bottle;
a second bottle having an interior and an aperture sized and threaded to mate with the first lid;
the second bottle is comprised of plastic;
the second bottle is opaque;
the threaded aperture of the second bottle has a diameter of 1.75 to 2.5 inches;
the interior of the second bottle has a volume of 20 to 40 cubic inches;
a sharps container warning label affixed to the first bottle;
unscrewing the first lid from the first bottle;
screwing the first lid onto a second bottle;
screwing a second lid onto the first bottle;
printing a first sharps container warning label;
affixing the first sharps container warning label to the first bottle;
printing a second sharps container warning label; and
affixing the second sharps container warning label to the second bottle.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense. While the disclosure is susceptible to various modifications and alternative constructions, certain illustrated examples thereof are shown in the drawings and have been described above in detail. It should be understood, however, that there is no intention to limit the disclosure to the specific forms disclosed, but on the contrary, the intention is to cover all modifications, alternative constructions, and equivalents falling within the spirit and scope of the disclosure.

What is claimed is:

1. A sharps disposal system comprising:
a first bottle having an interior and a threaded aperture; and
a first lid configured as a ratcheting safety lid, the first lid comprising:
a lower portion sized and threaded to mate with the threaded aperture of the first bottle, and having an off-center aperture; and;
an upper portion having an off-center aperture,
wherein the upper portion and the lower portion are arranged to enable the upper portion and the lower portion to rotate in one direction relatively to each other, such that the aperture of the upper portion can rotate to align with the aperture of the lower portion to provide access to the interior of the first bottle, and the aperture of the upper portion can rotate away from alignment with the aperture of the lower portion to close off access to the interior of the first bottle.

2. The system of claim 1 wherein the first bottle is comprised of plastic.

3. The system of claim 1 wherein the first bottle is opaque.

4. The system of claim 1 wherein the threaded aperture of the first bottle has a diameter of 1.75 to 2.5 inches.

5. The system of claim 1 wherein the interior of the first bottle has a volume of 20 to 40 cubic inches.

6. The system of claim 1 wherein the aperture of the lower portion has a maximum dimension of 0.25 to 0.75 inches.

7. The system of claim 1 wherein the aperture of the upper portion has a maximum dimension of 0.25 to 0.75 inches.

8. The system of claim 1
wherein the first lid comprises a set of ramped ratchet teeth and a set of cogs;
wherein the upper portion and the lower portion are arranged so that the set of ramped ratchet teeth and the set of cogs engage to permit the first lid to be screwed onto the first bottle;
wherein the upper portion and the lower portion are arranged so that the set of ramped ratchet teeth and the set of cogs permit the upper portion to rotate relatively to the lower portion without the first lid being unscrewed from the first bottle; and
wherein compression of the upper portion against the lower portion permits the first lid to be unscrewed from the first bottle.

9. The system of claim 1 further comprising:
a second lid sized and threaded to mate with the threaded aperture of the first bottle.

10. The system of claim 9 wherein the second lid is configured as a ratcheting safety lid and wherein the second lid does not have an aperture that can provide access to the interior of the first bottle.

11. The system of claim 1 further comprising:
a second bottle having an interior and an aperture sized and threaded to mate with the first lid.

12. The system of claim 11 wherein the second bottle is comprised of plastic, wherein the second bottle is opaque, wherein the threaded aperture of the second bottle has a diameter of 1.75 to 2.5 inches, and wherein the interior of the second bottle has a volume of 20 to 40 cubic inches.

13. The system of claim 1 further comprising:
a sharps container warning label affixed to the first bottle.

14. A method of sharps disposal, the method comprising:
rotating an upper portion of a first lid relatively to a lower portion of the first lid to provide access to an interior of a first bottle onto which the first lid is affixed;
placing a sharp object into the interior of the first bottle; and
rotating the upper portion relatively to the lower portion to close off access to the interior of the first bottle.

15. The method of claim 14 further comprising:
printing a first sharps container warning label; and
affixing the first sharps container warning label to the first bottle.

16. The method of claim 14 further comprising:
unscrewing the first lid from the first bottle.

17. The method of claim 14 further comprising:
screwing a second lid onto the first bottle.

18. The method of claim 14 further comprising:
screwing the first lid onto a second bottle.

19. The method of claim 18 further comprising:
printing a second sharps container warning label; and
affixing the second sharps container warning label to the second bottle.

20. A sharps disposal system comprising:
a first bottle having an interior and a threaded aperture, wherein the first bottle is comprised of plastic, wherein the first bottle is opaque, wherein the threaded aperture of the first bottle has a diameter of 1.75 to 2.5 inches, and wherein the interior of the first bottle has a volume of 20 to 40 cubic inches; and
a first lid configured as a ratcheting safety lid, the first lid comprising:
a lower portion sized and threaded to mate with the threaded aperture of the first bottle, and having an off-center aperture, wherein the aperture of the lower portion has a maximum dimension of 0.25 to 0.75 inches;
an upper portion having an off-center aperture, wherein the aperture of the upper portion has a maximum dimension of 0.25 to 0.75 inches;
wherein the upper portion and the lower portion are arranged to enable the upper portion and the lower portion to rotate in one direction relatively to each other, such that the aperture of the upper portion can rotate to align with the aperture of the lower portion to provide access to the interior of the first bottle, and the aperture of the upper portion can rotate away from alignment with the aperture of the lower portion to close off access to the interior of the first bottle;
wherein the first lid comprises a set of ramped ratchet teeth and a set of cogs;
wherein the upper portion and the lower portion are arranged so that the set of ramped ratchet teeth and the set of cogs engage to permit the first lid to be screwed onto the first bottle;
wherein the upper portion and the lower portion are arranged so that the set of ramped ratchet teeth and the set of cogs permit the upper portion to rotate relatively to the lower portion without the first lid being unscrewed from the first bottle; and
wherein compression of the upper portion against the lower portion permits the first lid to be unscrewed from the first bottle;
a second lid sized and threaded to mate with the threaded aperture of the first bottle, wherein the second lid is configured as a ratcheting safety lid and wherein the second lid does not have an aperture that can provide access to the interior of the first bottle; and
a second bottle having an interior and an aperture sized and threaded to mate with the first lid, wherein the second bottle is comprised of plastic, wherein the second bottle is opaque, wherein the threaded aperture of the second bottle has a diameter of 1.75 to 2.5 inches, and wherein the interior of the second bottle has a volume of 20 to 40 cubic inches.
